(19) United States
(12) Patent Application Publication
(10) Pub. No.: US 2019/0211370 A1
(43) Pub. Date: Jul. 11, 2019

(54) METHOD TO LABEL AS DEFECTIVE A MEASURE OF AN OPTICAL TRAP FORCE EXERTED ON A TRAPPED PARTICLE BY A TRAPPING LIGHT BEAM

(71) Applicant: IMPETUX OPTICS, S.L., Barcelona (ES)

(72) Inventors: Arnau FARRÉ FLAQUER, Barcelona (ES); Ferran MARSÀ SAMPER, Barcelona (ES)

(73) Assignee: IMPETUX OPTICS, S.L., Barcelona (ES)

(21) Appl. No.: 16/241,680

(22) Filed: Jan. 7, 2019

Related U.S. Application Data
(60) Provisional application No. 62/614,792, filed on Jan. 8, 2018.

(51) Int. Cl. C12Q 1/02; G01N 21/71; G01N 25/48

(52) U.S. Cl. CPC C12Q 1/02; G01N 33/48728; G01N 25/482; G01N 21/718

(57) ABSTRACT

A method to label as defective a measure of an optical-trap force, that is exerted on a trapped particle located inside a living, dispersive, viscoelastic medium, including operations of: (i) determining a calibration constant between the optical trap forces and the sensed voltages; (ii) determining a first calibration function of the frequency of the particle oscillation with the active-passive procedure; (iii) computing a second calibration function of the frequency as the quotient between the calibration constant and the first calibration function; (iv) computing an energy function of the frequency as the product of the thermal energy of the trapped particle and the second calibration function; (v) checking whether the energy function converges to the thermal energy of the trapped particle as the frequency increases; (vi) if there is no such convergence, then label as defective the measure of the optical-trap force.

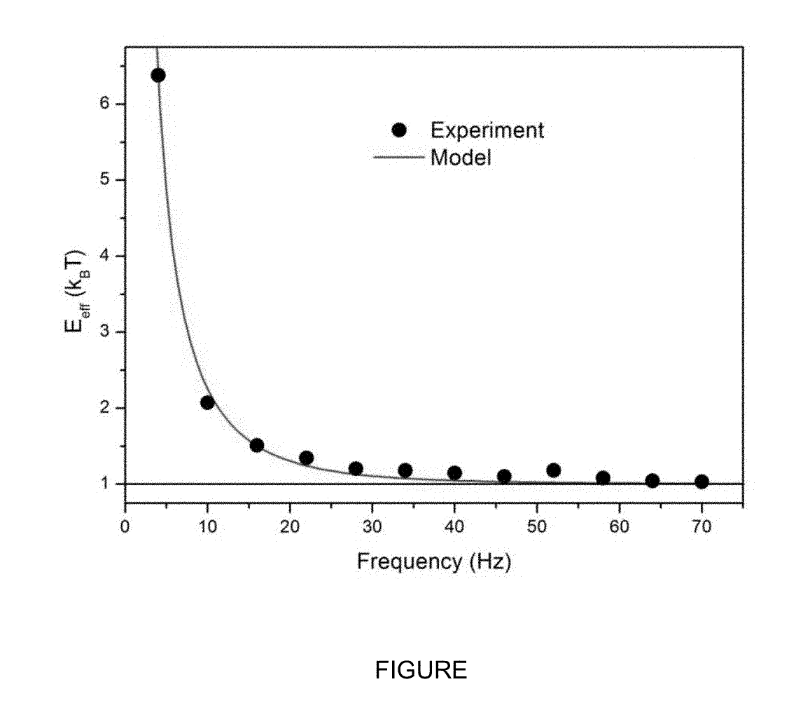

The present disclosure relates to a method to label as defective a measure of an optical-trap force exerted on a trapped particle by a trapping light beam, the particle being located inside a living, dispersive, viscoelastic medium.

### BACKGROUND

In vitro force measurements on single biomolecules and other biological structures can be accomplished by using optical tweezers to trap them, but performing such force measurements in their natural environment, e.g. the cell cytoplasm, is usually much harder because the normal calibration procedures for single beam optical traps only apply to simple liquids, the respective viscosities of which are known. In contrast, these quantities are often unknown when the probe is inside the cytoplasm or another viscoelastic medium.

Nevertheless, in 2010 Mario Fischer et al. proposed a procedure to calibrate a single beam optical tweezers in situ within a viscoelastic medium (“Active-passive calibration of optical tweezers in viscoelastic media”, Review of Scientific Instruments 81, 015103). An optically trapped particle performs Brownian motion in a quasiharmonic potential. Knowledge of the stiffness parameter κ of the quasiharmonic potential and the position x allows for measuring or exerting prescribed forces of order pN. In a calibration procedure the trapped particle is driven by forces of known characteristics and its trajectory is measured; such driving is denoted as passive. Another type of calibration requires a controlled motion of the trapping laser with respect to the sample chamber (or vice versa); this type of driving is denoted as active.

The Fischer team applied the fluctuation-dissipation theorem (FDT) to combine passive and active measurements in order to find the spring constant κ that characterizes the strength of the optical trap (trap stiffness), the response function χ(f) (which is a function of the frequency f of the oscillating motion), etc. However, this active-passive procedure is based on two assumptions which even in the best case scenario are only approximately verified within the cytoplasm:
The active-passive procedure is based on linear response theory in continuous media.
All particle motion takes place within the harmonic region of the trapping potential.

The optical force exerted by a laser trap (e.g. optical tweezers) in a cell can be measured through a second procedure, which is not tied to these assumptions. One approach is to use the deflection of the trapping laser caused by the trapped particle to determine the rate of momentum change with a photodetector (e.g. a position sensitive detector, PSD) located at the back focal plane, or at an optical equivalent thereof, of a lens that is positioned so as to collect all the light scattered by the trapped object. This direct momentum procedure has clear advantages over the former one as no in situ calibration is required. The factor αₛₑₙₛₒᵣ used to convert the output voltage from the sensor to picoNewton (pN) units is valid regardless of the experimental conditions. The constant αₛₑₙₛₒᵣ is obtained from a macroscopic calibration of the instrument which is the result of the measurement of several parameters, such as the photodetector radius or the instrument transmittance, among others.

One of the advantages of this macroscopic or direct calibration procedure compared to the active-passive one is that force calibration is not affected by biological activity. M. Fischer et al. already stated in their work that an important concern when applying the active-passive method to living cells is the presence of bioactive processes which renders the FDT invalid in certain frequency ranges. In fact, using the violation of the FDT, Wylie W. Ahmed et al. (“Active mechanics reveal molecular-scale force kinetics in living oocytes”, arXiv:1510.08299v2 [physics.bio-ph] 8 Nov. 2016) noted that in living cells the presence of biochemical activity gives rise to active forces (e.g. a non-equilibrium process) driven by energy consuming processes, so that the force driving the motion of particles in a living cell has two contributions: (1) a passive (purely thermal) contribution described by classical equilibrium physics; (2) an active contribution that is biochemically regulated and cannot be understood via equilibrium physics.

To quantify the non-equilibrium activity in a biologically active material it is instructive to consider the effective energy, which is a measure of how far the system is from thermal equilibrium, i.e., the greater the difference between the effective energy and the thermal (equilibrium) energy, the farther the system is from thermal equilibrium, since the thermal energy is precisely the internal energy present in a system in a state of thermodynamic equilibrium by virtue of its temperature.

Ultimately, the main concern when dealing with the question of measuring forces inside cells layers or biological tissues with the direct momentum procedure is whether the measured forces correspond solely to those applied to the trapped object or, by contrast, there are contributions from light scattered at other planes. Tissue structures extending both above and below the trapped sample may scatter light and modify the propagation of the beam, resulting in contaminated force measurements.

### SUMMARY

The present developments provide methods to assess the reliability of a measure of the force exerted on a particle trapped inside a cell by a trapping light beam, e.g. an optical tweezer.

In a first aspect, such a method, for being able to label as defective a measure of an optical-trap force exerted on a trapped particle located inside a viscoelastic medium, may include the operations of:
determining a calibration constant with the known macroscopic direct procedure (i.e. the momentum direct procedure);
determining a first calibration function of the frequency of the trapped particle oscillation with the known active-passive procedure, the first calibration function including the thermal energy of the trapped particle as a multiplicative factor;
computing a second calibration function of the frequency of the trapped particle oscillation as the quotient between the calibration constant and the first calibration function;
computing an energy function of the frequency of the trapped particle oscillation as the product of the thermal energy of the trapped particle and the second calibration function;
checking whether the energy function converges to the thermal energy of the trapped particle as the frequency of the oscillation thereof increases;
if there is no such convergence, then label as defective the measure of the optical-trap force.

The energy function may actually be the effective energy of the trapped particle. It can be inferred, then, that the non-convergence of the effective energy to the thermal energy is a sign of the prevalence of out-of-focus scattering that distorts the deflection of the light beam caused by the trapped particle and thus contaminates the force measurement.

The viscoelastic medium shall usually be the inside of a living cell, which is likely populated with dispersive (i.e. scattering) structures. The viscoelastic medium may be a living and dispersive medium.

The calibration constant determined with the macroscopic direct procedure is the proportionality constant between the optical trap force and the voltage measured with a back-focal-plane interferometer in the framework of the known direct momentum procedure.

The present developments may also provide methods to detect out-of-focus scattering when measuring an optical-trap force exerted by a trapping light beam on a trapped particle.

In a second aspect, such a method, for being able to reveal the presence of disrupting out-of-focus tissue structures when a measurement of an optical-trap force exerted by a trapping light beam on a trapped particle is performed, may include the operations of:
determining a calibration constant with the known macroscopic direct procedure;
determining a first calibration function of the frequency of the trapped particle oscillation with the known active-passive procedure, the first calibration function including the thermal energy of the trapped particle as a multiplicative factor;
computing a second calibration function of the frequency of the trapped particle oscillation as the quotient between the calibration constant and the first calibration function;
computing an energy function of the frequency of the trapped particle oscillation as the product of the thermal energy of the trapped particle and the second calibration function;
checking whether the energy function converges to the thermal energy of the trapped particle as the frequency of the oscillation thereof increases;
if there is no such convergence, then mark the presence of disrupting out-of-focus tissue structures that scatter the light beam.

These operations are the same as those present in the first aspect, but are directed to a somewhat different goal. Other features related to the first aspect method may also be analogously related to the second aspect method.

In a third aspect, an apparatus to perform any of the previous methods may include a photodetector to capture the photons scattered by the trapped particle.

Further advantages, properties, aspects and features of the present disclosure may be derived from both the appended claims and the below-described examples. The above-described features and/or the features disclosed in the claims and/or in the following description of examples and clauses can, if required, also be combined with one another even if this is not expressly described in detail.

### BRIEF DESCRIPTION OF THE DRAWINGS

Non-limiting examples of the present disclosure will be described in the following, with reference to the appended drawings, in which the sole FIGURE, the FIGURE, is a graph that plots the effective energy against the frequency of the particle oscillations.

### DETAILED DESCRIPTION OF EXAMPLES

Taking advantage of the difference in nature of the two aforementioned calibrations (the macroscopic direct calibration and the microscopic active-passive calibration), herein disclosed are methods to determine the presence of the out-of-focus scattering that is fairly typical in dispersive samples. The integration of the macroscopic and microscopic approaches provides for detection of the existence of beam momentum changes outside the trapping region.

The methods exploit the complementarity between the two approaches using each one as a benchmark for the other one. On the one hand, the sensitivity of the active-passive calibration to the specific conditions of the experiment allows for identifying and quantifying the biological activity at low frequencies (<100 Hz, approx.) so as to have evidence of the reliability of the calibration. On the other hand, the validated robustness of the sensor's momentum response in the packed cytoplasm of cells provides a reference for the in situ calibrations at high frequencies (>100 Hz, approx.). Both approaches combined together give a procedure to discard results affected by out-of-focus momentum changes.

The methods may include the following operations:
1. A first operation may involve reducing the sources of noise, such as stage drifts or laser pointing fluctuations, below a critical limit where non-equilibrium effects may become noticeable (i.e. significant).
2. Macroscopic direct calibration (momentum procedure): calibration constant αₛₑₙₛₒᵣ (force=αₛₑₙₛₒᵣ·Voltage).
3. Active-passive calibration: calibrate αₜᵣₐₚ(ω) as

$$\frac{2 {k}_{B} T}{ω {P}^{V} ( ω )} {Im ( {\tilde{V}}_{dr} / {\tilde{x}}_{s} )}_{ω}$$

for different frequencies fin the range 1 Hz-1 kHz (or at least 1-100 Hz or 1-200 Hz) with the same trapped particle, where ω is the angular frequency 2πf, T is the absolute temperature, V_{dr} and Xₛ are the Fourier transform of the recorded output voltage and the stage displacement, respectively, and P^{V} is the power spectral density of the passive motion of the sample as measured by the detection system. If oscillations of the particle are induced by the motion of the trap, X_{S} is replaced by X_{L}, the Fourier transform of the laser displacement, and the preceding equation includes a minus (−) sign. By using the equation, the force calibration can be obtained even if the sample is outside the linear region of the force. If necessary, one just needs to measure the position-voltage curve and multiply the calibration by (β_{dr}/β_{P})², where β represents the position calibrations for the driving signal and for passive spectrum, respectively.
4. Compute the non-equilibrium activity through the effective energy obtained as E_{eff} (f)=Eₜₕₑᵣₘ·(αₛₑₙₛₒᵣ/αₜᵣₐₚ(ω), where Eₜₕₑᵣₘ=k_{B}T.
5. Plot E_{eff} (f) as a function of frequency (see the FIGURE). Its value should be several times that of Eₜₕₑᵣₘ in the range of 1-10 Hz and decrease monotonically until approximately 100-200 Hz, where it should converge to Eₜₕₑᵣₘ.
6. If this convergence does not show up, the force measurements may be contaminated by out-of-focus structures and should be discarded.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow. If reference signs related to drawings are placed in parentheses in a claim, they are solely for attempting to increase the intelligibility of the claim, and shall not be construed as limiting the scope of the claim.

1. A method to label as defective a measure of an optical-trap force exerted on a trapped particle by a trapping light beam, the particle being located inside a viscoelastic medium, the method comprising the operations of:
determining a calibration constant with the known macroscopic direct procedure;
determining a first calibration function of the frequency of the trapped particle oscillation with the known active-passive procedure, the first calibration function including the thermal energy of the trapped particle as a multiplicative factor;
computing a second calibration function of the frequency of the trapped particle oscillation as the quotient between the calibration constant and the first calibration function;
computing an energy function of the frequency of the trapped particle oscillation as the product of the thermal energy of the trapped particle and the second calibration function;
checking whether the energy function converges to the thermal energy of the trapped particle as the frequency of the oscillation thereof increases;
if there is no such convergence, then labelling as defective the measure of the optical-trap force.

2. The method of claim 1, the optical trap being a single-beam optical tweezers.

3. The method of claim 1, the particle being located within a biological tissue.

4. The method of claim 3, the particle being located within a cell.

5. The method of claim 4, the particle being located within a cell cytoplasm.

6. The method of claim 2, the particle being located within a cell.

7. The method of claim 6, the particle being located within a cell cytoplasm.

8. The method of claim 1, the setup to determine the calibration constant comprising a photodetector, and the calibration constant being derived from one or more of the photodetector radius and other parameters.

9. The method of claim 8, the calibration constant being derived from one or more of the transmittance of said setup and other parameters.

10. The method of claim 2, the setup to determine the calibration constant comprising a photodetector, and the calibration constant is derived from one or more of the photodetector radius and other parameters.

11. The method of claim 10, the calibration constant being derived from one or more of the transmittance of said setup and other parameters.

12. The method of claim 1, comprising an operation that is prior to the stated operations, said prior operation including limiting the stage drifts below a threshold that renders significant the non-equilibrium effects.

13. The method of claim 2, comprising an operation that is prior to the stated operations, said prior operation including limiting the stage drifts below a threshold that renders significant the non-equilibrium effects.

14. The method of claim 1, comprising an operation that is prior to the stated operations, said prior operation including limiting the laser pointing fluctuations below a threshold that renders significant the non-equilibrium effects.

15. The method of claim 2, comprising an operation that is prior to the stated operations, said prior operation including limiting the laser pointing fluctuations below a threshold that renders significant the non-equilibrium effects.

16. A method to reveal the presence of disrupting out-of-focus tissue structures when a measurement of an optical-trap force exerted by a trapping light beam on a trapped particle is performed, comprising the operations of:
determining a calibration constant with the known macroscopic direct procedure;
determining a first calibration function of the frequency of the trapped particle oscillation with the known active-passive procedure, the first calibration function including the thermal energy of the trapped particle as a multiplicative factor;
computing a second calibration function of the frequency of the trapped particle oscillation as the quotient between the calibration constant and the first calibration function;
computing an energy function of the frequency of the trapped particle oscillation as the product of the thermal energy of the trapped particle and the second calibration function;
checking whether the energy function converges to the thermal energy of the trapped particle as the frequency of the oscillation thereof increases;
if there is no such convergence, then mark the presence of disrupting out-of-focus tissue structures that scatter the light beam.

17. An apparatus to perform the method of claim 1, comprising a photodetector.

18. The apparatus of claim 17, comprising a single laser source to emit the trapping light beam.

19. The apparatus of claim 17, comprising a back-focal-plane interferometer.

20. The apparatus of claim 18, comprising a back-focal-plane interferometer.
